(19) United States
(12) Patent Application Publication
(10) Pub. No.: US 2023/0194489 A1
(43) Pub. Date: Jun. 22, 2023

(54) AIR QUALITY MONITORING DEVICE AND SYSTEM

(71) Applicant: AMBISENSE LTD., Dublin (IE)

(72) Inventors: Stephen MCNULTY, Dublin (IE); Thomas ARCHER, Dublin (IE)

(21) Appl. No.: 17/926,121

(22) PCT Filed: May 18, 2021
(86) PCT No.: PCT/EP2021/063214

§ 371 Date: Nov. 17, 2022

(30) Foreign Application Priority Data
May 18, 2020 (GB) 2007340.9

(51) Int. Cl. G01N 33/00; G06N 20/00

(52) U.S. Cl. CPC G01N 33/0031; G06N 20/00

(57) ABSTRACT

An improved air quality monitoring device provides accurate and reliable predictions regarding likelihood for microbial growth on building walls. The present invention comprises a housing adapted to affix the device to a building wall, a first sensor array placed in close proximity to the wall, a second sensor array and a processing means operatively connected to the first sensor array and the second sensor array. The first sensor array provides inputs such as relative humidity at the surface of the wall, the wall temperature and wall pressure. The second sensor array provides inputs such as relative humidity, temperature pressure and quality of ambient air. Using inputs from the first sensor array and the second sensor array, the processing means generates an output representative of the likelihood of microbial growth and the likelihood that the wall moisture is caused by condensation. A system of networked monitoring devices is also described.

### FIELD

The present disclosure relates to a monitoring device and system, and more particularly to an air quality monitoring device which enables prevention and/or detection of microbial growth on wall interiors.

### BACKGROUND

There are many factors which influence air quality, the major ones being temperature, carbon dioxide content, volatile organic compounds (VOC), microbial spores (for example mold spores) and humidity. Growth of fungi, especially certain types of molds in residential and commercial buildings needs special consideration because of its effect on the health of the occupants of the building, and the difficulty and time taken for dealing with such fungal growth. Once established, mold colonies quickly spread and are difficult to control. Common molds such as Stachybotrys, Trichoderma and Penicillium causes health issues such as dermatitis, lung infections, high fever and allergies.

One of the contributors to microbial (mold) growth on building walls is relative humidity in ambient air. Most conventional air monitoring devices use relative humidity of ambient air as the major parameter for ascertaining conditions for likelihood of mold growth. However, recent research proves that relative humidity in ambient air might not be the most accurate and reliable metric which could be used to ascertain probability of mold growth. Factors such as relative humidity measured in equilibrium with the surface (for example, building walls) which has potential for mold growth, provide significantly higher correlation with likelihood of mold growth.

In addition, conventional air monitoring devices do not provide information on where moisture in the walls may be coming from. By measuring the relative humidity and temperature of the air the dewpoint can be determined, this is the temperature below which condensation will occur, if the wall temperature drops below the dewpoint condensation occurs. Through correlating the response of the wall moisture with condensation, a monitoring solution can assess if wall moisture is due to condensation or comes from an alternative source e.g. penetrating or rising damp.

Examples in the art include Chinese Patent Publication Number CN207717125 (Bosch GMBH) which provides an independent analysis of data from the two sensor benches allowing the prediction of mould. German Patent Publication DE10214107690 improves on CN207717125 by providing synergy between the sensor benches to diagnose where the moisture comes from. However these systems are proven not to be very accurate and complex to make and implement.

There is therefore an unresolved and unfulfilled need for an improved air quality monitoring device which can provide more reliable and accurate predictions regarding likelihood for microbial growth in buildings, and this forms an objective of the present invention.

### SUMMARY

The present invention relates to an improved air quality monitoring device and system, as set out in the appended claims.

In one embodiment the device as per the present invention comprises a housing which is adapted to affix the device onto a building wall. The housing further comprises a rear plate and a front plate.

In one embodiment the rear plate is placed in contact with the wall and the front plate is disposed adjacent to the rear plate. At least one through-hole is formed on both the rear plate and the front plate.

In one embodiment a first sensor array and a second sensor array are respectively mounted on the rear plate and the front plate, such that both the sensor arrays are placed concentric to the respective through holes.

In one embodiment the first sensor array is placed in close proximity to, and in equilibrium with the building wall. The first sensor array includes a plurality of temperature sensors, pressure sensors and humidity sensors and measures the relative humidity of the wall surface, wall temperature and pressure.

In one embodiment the second sensor array includes a plurality of pressure sensors, temperature sensors, humidity sensors, carbon dioxide sensors and volatile organic compound sensors and measures the relative humidity, pressure, temperature and quality of ambient air.

The device as per the present invention also comprises a processing means disposed in the housing. The processing means is operatively connected to the first sensor array and the second sensor array. The processing means processes inputs from the first sensor array and the second sensor array and provides an output representative of the likelihood of mold growth. The processor can also be configured to detect or predict a microbial or mold growth on the wall.

In one embodiment the air quality monitoring device as per the present invention further comprises a thermal camera which enables to identify thermal bridges or cold patches within the building wall and hence helps in optimal placement of the air monitoring device.

In one embodiment the air monitoring device as per the present invention accurately identifies conditions inducive for mold growth. This is enabled by placing sensors in equilibrium with the building walls and thereby using inputs such as relative humidity at the surface of the wall and wall temperature, in addition to inputs related to ambient air relative humidity, ambient air quality and ambient air temperature and pressure.

In one embodiment the second sensor array is configured to measure the wall temperature, and when the wall temperature falls below a dewpoint condensation occurs, the device is configured to correlate condensation with equilibrium relative humidity, such that the degree to which wall moisture is driven by condensation can be ascertained.

In one embodiment the rear plate comprises an airtight seal capturing a volume of air behind the device, said captured volume of air is in both thermal and moisture equilibrium with the wall.

In one embodiment the seal comprises a polyurethane gel gasket positioned between the device and the mounting surface to ensure the integrity of the airtight seal.

In one embodiment a putty compound is positioned to ensure that the wall humidity sensor is isolated from the rest of the device and forms an airtight seal into the volume of air encapsulated by the device and gasket at the wall.

In another embodiment there is provided an air quality monitoring system comprising a plurality of air quality monitoring devices as claimed in any preceding claim, wherein each monitoring device is configured with bi-directional communication to send and receive information, the monitoring system comprising a computing device adapted to:
send and receive information to and from each monitoring device;
a module configured to collect and store the received data from at least one monitoring device;
a module to receive contextual data from one or more sources;
processing the received data from the monitoring devices and contextual data; and
outputting a prediction data for a time when mold or other bacteria will form in the vicinity of a monitoring device.

In one embodiment the one or more sources comprises one or more of the following: a local weather source; a home heating source associated with a monitoring device location; a database source of stored historical data; a ventilation source or an air conditioning source.

In one embodiment a machine learning algorithm is configured to output the prediction data for a time when mold or other bacteria will form.

In one embodiment the machine learning algorithm receives inputs from at least one monitoring device and contextual data from one or more sources.

In one embodiment the processor is configured, on generation of the prediction data, to generate a control signal to at least one source to control the ambient temperature conditions in the vicinity of a known monitoring device location. Ideally the control source comprises a smart heating system configured to control the temperature of an area in which a monitoring device is positioned.

There is also provided a computer program comprising program instructions for causing a computer program to carry out the above method which may be embodied on a record medium, carrier signal or read-only memory.

### BRIEF DESCRIPTION OF DRAWINGS

The invention will be more clearly understood from the following description of an embodiment thereof, given by way of example only, with reference to the accompanying drawings, in which:

FIG. 1 illustrates a schematic diagram of an air quality monitoring device as per a preferred embodiment of the present invention;

FIG. 2 is a graphical representation illustrating the relationship between relative humidity, and difference between wall temperature and ambient air temperature; and

FIG. 3 illustrates a network of air quality monitoring devices connected to a remote server or computing device according to one embodiment of the present invention.

### DETAILED DESCRIPTION OF INVENTION

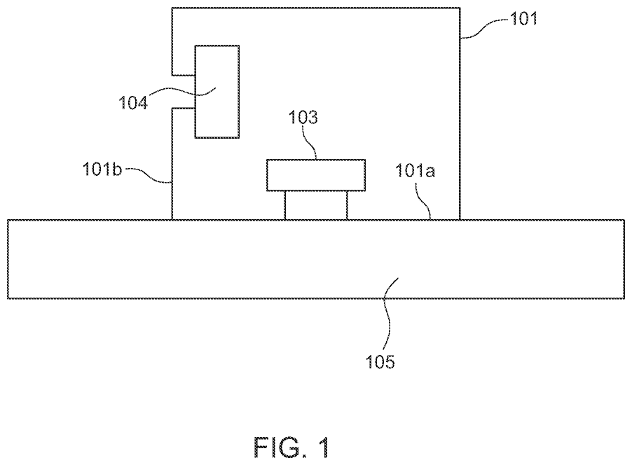

FIG. 1 is a schematic diagram of a preferred embodiment of the present invention. Referring to FIG. 1, the air quality monitoring device comprises a housing 101 adapted to affix the device onto a building wall 105. The housing further comprises a rear plate 101a which is placed in contact with the wall 105 and a front plate 101b disposed adjacent to the rear plate 101a. Both the rear plate 101a and the front plate 101b have at least one through-hole formed on their surfaces.

A first sensor array 103 is mounted on the rear plate 101a such that it is placed concentric with the through-hole. This enables the first sensor array 103 to be placed in close proximity to the wall 105 and to measure parameters in equilibrium with the wall 105 surface. The first sensor array 103 comprises a plurality of humidity sensors, temperature sensors and pressure sensors, and measures the relative humidity of the wall surface, the wall temperature and the wall pressure.

A second sensor array 104 is mounted on the front plate 101b such that it is placed concentric with the through hole(s) formed on the front plate 101b.

The second sensor array comprises a plurality of humidity sensors, temperature sensors, pressure sensors, carbon dioxide sensors and volatile organic compound sensors, and measures the relative humidity of the ambient air, ambient air temperature, ambient air pressure and ambient air quality.

A processing means disposed within the housing 101 is operatively connected to the first sensor array 103 and the second sensor array 104. The processing means processes outputs from the first sensor array 103 and computes the dewpoint temperature (T_{dp}). The second sensor array 104 measures the wall temperature. When the wall temperature falls below the dewpoint condensation occurs. Through correlating condensation with equilibrium relative humidity, measured with sensor array 104, the degree to which wall moisture is driven by condensation can be ascertained. Which in turn helps in prediction of damp and which in turn drives micro bacterial growth. In a preferred embodiment of the present invention the processing means used is a Long Range (LoRa) 868 MHz RFM95 communication module.

The wall sensor comprises an airtight seal capturing a volume of air behind the device which is in both thermal and moisture equilibrium with the mounting surface. The seal is created using a polyurethane gel gasket between the device and the mounting surface ensuring a strong, airtight seal. Internally, a putty compound ensures that the wall humidity sensor is isolated from the rest of the device and forms an airtight seal into the volume of air encapsulated by the device and gasket at the wall.

The wall temperature sensor is a separate sensor placed in contact with the wall allowing accurate temperature measurement.

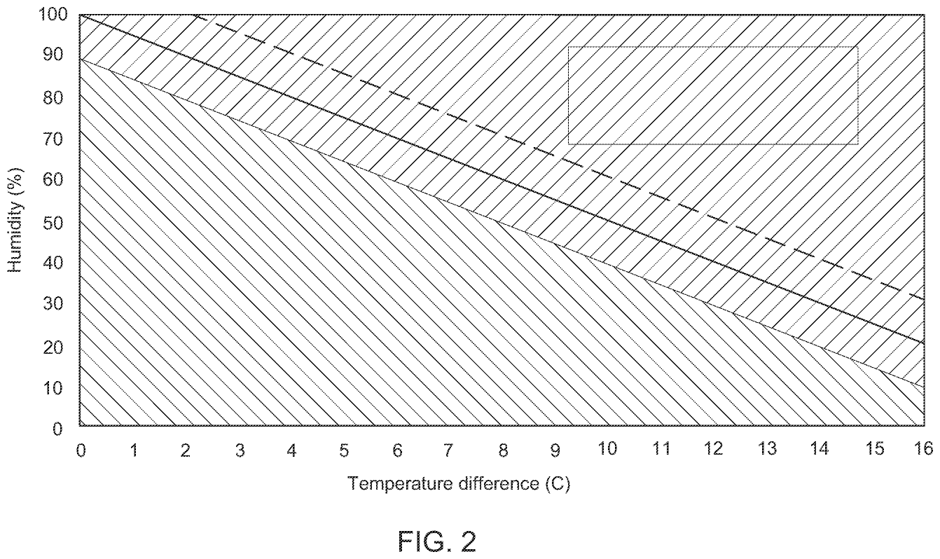

FIG. 2 is a graphical representation illustrating the relationship between relative humidity of the ambient air, and the difference in wall temperature (T_{wall}) and ambient air temperature (Tₐᵢᵣ). Greater the difference in temperature, lesser shall be the relative humidity to prevent condensation. As shown in upper half of the figure (dark shading), condensation occurs beyond a threshold value of relative humidity, which in turn creates conducive conditions for microbial growth. Relative humidity values corresponding to temperature differences in the lower half of the figure (light shading) are considered safe and does not lead to condensation.

The air quality monitoring device as per the present invention further comprises a thermal camera. The placement of the device is one of the key factors for its optimum functioning. The device needs to be placed on the relatively coldest patch of the building wall or near windows where thermal bridges may occur. This is enabled by the thermal camera which identifies the coldest spot using thermal imagery.

Location of the monitoring devices are optimised through conducting a thermal scan of the installation area. This thermal model provides contextual data to allow a single device to monitor to infer the behaviour of a larger space. The thermal camera can be integrated into the device or alternatively is a standalone thermal camera device that can be used to determine the optimum positioning of the air monitoring quality device in a room, a corridor or enclosed area.

The present invention can work on two principles:
1) Direct measurement of the equilibrium relative humidity (ERH) and prolonged periods of high ERH are strongly correlated with micro bacterial growth.
2) Identification of the cause of high ERH. If the wall temperature falls bellow the dewpoint condensation occurs on the wall. Through correlating the ERH with the proportion of time water is condensing on a surface provides a measure of whether the moisture present in the wall is due to condensation or some other mechanism.

Readings from the device are recorded every 15 minutes and can be viewed by stakeholders or third party in a user interface operatively connected to the processing means.

Preventive measures against microbial growth shall be initiated in one instance if condensation is occurring or ERH>60% for more than 50% of the time on a weekly basis.

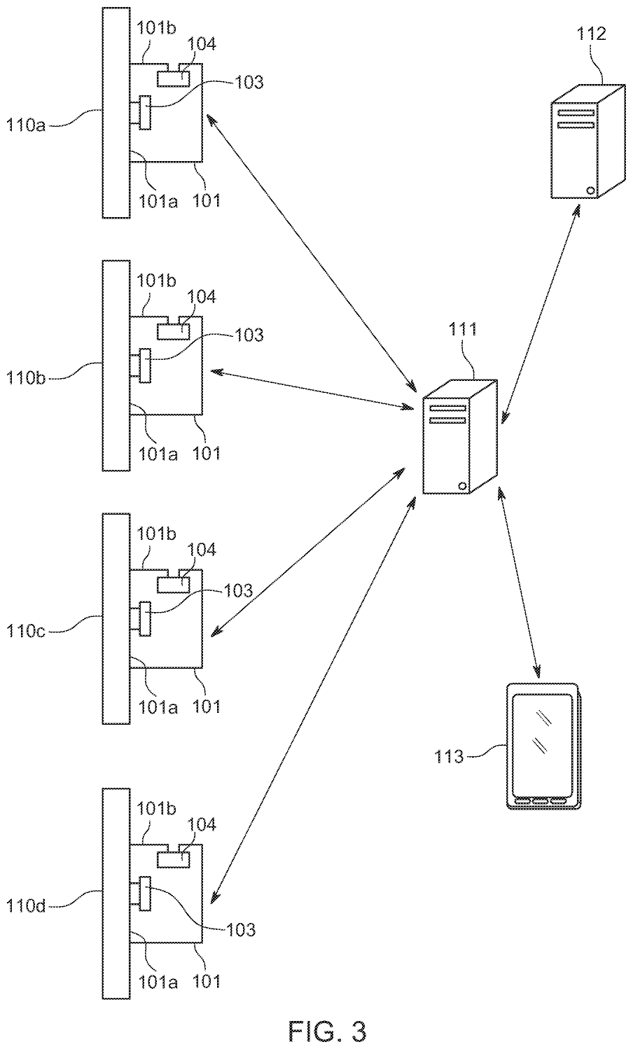

FIG. 3 illustrates a network of air quality monitoring devices (110a, 110b, 110c & 110d) connected to a remote server or computing device (111) according to another embodiment of the invention. The configuration of each device is the same as that described with the monitoring device of FIG. 1. The network comprises a plurality of monitoring devices, with four shown but not limited, which are configured to receive and send data to a central server of computing device (111). Each monitoring device (110a, 110b, 110c & 110d) is configured with bi-directional communication to send and receive information. The computing device (111) can send and receive information to and from each monitoring device (110a, 110b, 110c & 110d).

The computing device (111) has a module configured to collect and store the received data from at least one monitoring device. The same, or a separate module, is configured to receive contextual data from one or more sources (112). The computing device processes the received data from the monitoring devices and contextual data from the at least one data source (112). The computing device (111) can then output a prediction data indicating a time when mold or other bacteria will or is estimated to form in the vicinity of a monitoring device (110a, 110b, 110c & 110d). This information can be outputted to a screen or a mobile computing device (113), such as a smart phone or tablet.

It will be appreciated that the network system can combine data from multiple devices, external sources and provide feedback to heating, ventilation, and air conditioning systems. The communication can be facilitated by low power 2-way radio communication.

Data from the devices (110a, 110b, 110c & 110d) is collected and modelled on a central server along with contextual data from other sources (112) including but not limited to local weather, occupancy, and/or heating, ventilation, and air conditioning systems. The correlation between the contextual sources is analysed through machine learning to improve the prediction for:
a) the time to mould formation
b) the diagnostics for the observed moisture and allowing differentiation to be made for penetrating vs rising damp.

The processor is configured, on generation of the prediction data, to generate a control signal and transmit to at least one source (111) to control the ambient temperature conditions in the vicinity of a known monitoring device location. Ideally the control source comprises a smart heating system configured to control the temperature of an area in which a monitoring device is positioned. Communication of the control signal can be provided to the heating, ventilation, and/or air conditioning system. Controlling the environmental conditions to mitigate damp and mould formation. This removes the need for an always-on approach to preventing mould.

Communication of the recorded data from one or more monitoring devices to a central computing resource provides an improvement in the predictive and modelling capacity by combining data from other devices as well as external driving factors for example heating systems. The communications also provide a feedback loop to HVAC systems and the like to maintain a mold-free environment.

Data from multiple devices provides feedback to a learning algorithm. The inter-device synergy is used to build up knowledge of the wetting and drying times as a function of environmental condition improving the predictive capacity of the system.

The embodiments in the invention described with reference to the drawings comprise a computer apparatus and/or processes performed in a computer apparatus. However, the invention also extends to computer programs, particularly computer programs stored on or in a carrier adapted to bring the invention into practice. The program may be in the form of source code, object code, or a code intermediate source and object code, such as in partially compiled form or in any other form suitable for use in the implementation of the method according to the invention. The carrier may comprise a storage medium such as a memory stick or hard disk. The carrier may be an electrical or optical signal which may be transmitted via an electrical or an optical cable or by radio or other means.

In the specification the terms “comprise, comprises, comprised and comprising” or any variation thereof and the terms include, includes, included and including” or any variation thereof are considered to be totally interchangeable and they should all be afforded the widest possible interpretation and vice versa.

Although the present invention has been described with reference to specific embodiments, this description is not meant to be construed in a limiting sense. Various modifications of the disclosed embodiments, as well as alternate embodiments of the subject matter, will become apparent to persons skilled in the art upon reference to the description of the subject matter. It is therefore contemplated that such modifications can be made without departing from the spirit or scope of the present invention as defined.

The invention is not limited to the embodiments hereinbefore described but may be varied in both construction and detail.

1. An air quality monitoring device (110) comprising:
a housing (101) configured to affix the device to an interior space of a wall (105), the housing (101) having a rear plate (101a) placed in contact with the wall (105) and a front plate (101b) disposed adjacent to the rear plate (101a);
a first sensor array (103) mounted on the rear plate (101a), the first sensor array (103) provides a plurality of output signals representative of at least one of relative humidity at the wall surface, wall temperature and wall pressure;
a second sensor array (104) mounted on the front plate 101b, the second sensor array (104) provides a plurality of output signals representative of at least one of ambient air temperature, ambient air pressure, ambient air quality and ambient air relative humidity; and
a processor means disposed within the housing (101) and operatively connected to the first sensor array (103) and the second sensor array (104) wherein the processor is configured to detect or predict a microbial or mold growth on the wall.

2. The air quality monitoring device as claimed in claim 1, wherein the first sensor array (103) comprises a plurality of pressure sensors, temperature sensors and humidity sensors.

3. The air quality monitoring device as claimed in claim 1, wherein the second sensor array (104) comprises a plurality of pressure sensors, temperature sensors, humidity sensors, carbon dioxide sensors and volatile organic compound sensors.

4. The air quality monitoring device as claimed in claim 1 wherein the second sensor array (104) is configured to measure the wall temperature, and the processor calculates when the wall temperature falls below a dewpoint condensation occurs, the device is configured to correlate condensation with equilibrium relative humidity, such that the degree to which wall moisture is driven by condensation can be ascertained.

5. The air quality monitoring device as claimed in claim 1, wherein the device further comprises a thermal camera.

6. The air quality monitoring device as claimed in claim 1, wherein the processing means comprises a long range (LoRa) 868 MHz RFM95 communication module.

7. The air quality monitoring device as claimed in claim 1, wherein the rear plate (101a) has formed on it at least one through-hole and wherein the first sensor (103) array is mounted concentric to the through-hole.

8. The air quality monitoring device as claimed in claim 1, wherein the front plate (101b) has formed on it at least one through-hole and wherein the second sensor array (104) is concentric to the through hole.

9. The air quality monitoring device as claimed in claim 1 wherein the rear plate (101a) comprises an airtight seal capturing a volume of air behind the device, said captured volume of air is in both thermal and moisture equilibrium with the wall (105).

10. The air quality monitoring device as claimed in claim 9 wherein the seal comprises a polyurethane gel gasket positioned between the rear plate (101a) and the mounting surface to ensure the integrity of the airtight seal.

11. The air quality monitoring device as claimed in claim 1 comprising a putty compound positioned to ensure that the wall humidity sensor is isolated from the rest of the device and forms an airtight seal into the volume of air encapsulated by the device and gasket at the wall.

12. An air quality monitoring system comprising a plurality of monitoring devices (110a, 110b, 110c & 110d) as claimed in any preceding claim, wherein each monitoring device is configured with bi-directional communication to send and receive information, the monitoring system comprising a computing device (111) adapted to:
send and receive information to and from each monitoring device (110a, 110b, 110c & 110d);
a module configured to collect and store the received data from at least one monitoring device;
a module to receive contextual data from one or more sources (112);
processing the received data from the monitoring devices (110a, 110b, 110c & 110d) and contextual data from at least one source (112); and
outputting a prediction data for a time when mold or other bacteria will form in the vicinity of a monitoring device.

13. The air quality monitoring system of claim 12 where the one or more sources comprises one or more of the following: a local weather source; a home heating source associated with a monitoring device location; a database source of stored historical data; a ventilation source or an air conditioning source.

14. The air quality monitoring system of claim 12 wherein a machine learning algorithm is configured to output the prediction data for a time when mold or other bacteria will form.

15. The air quality monitoring system as claimed in claim 14 wherein the machine learning algorithm receives inputs from at least one monitoring device and contextual data from one or more sources.

16. The air quality monitoring system as claimed claim 12 wherein the processor is configured, on generation of the prediction data, to generate a control signal to at least one source to control the ambient temperature conditions in the vicinity of a known monitoring device location.
